(19) United States
(12) Patent Application Publication
(10) Pub. No.: US 2022/0220436 A1
(43) Pub. Date: Jul. 14, 2022

(54) ALGINATE DIALDEHYDE-COLLAGEN HYDROGELS AND THEIR USE IN 3D CELL CULTURE

(71) Applicant: FRIEDRICH-ALEXANDER UNIVERSITÄT ERLANGEN-NÜRNBERG, ERLANGEN (DE)

(72) Inventors: STEFANIE KLOSTERMEIER, ERLANGEN (DE); KARL MESSLINGER, BUCKENHOF (DE); ROBERTO DE COL, REDNITZHEMBACH (DE); ALDO ROBERTO BOCCACCINI, ERLANGEN (DE); THOMAS DISTLER, NUERNBERG (DE); RAINER DETSCH, ERLANGEN (DE)

(21) Appl. No.: 17/614,180

(22) PCT Filed: Jun. 4, 2020
(86) PCT No.: PCT/EP2020/065537

§ 371 Date: Nov. 24, 2021

(30) Foreign Application Priority Data
Jun. 7, 2019 (EP) 19179014.6

(51) Int. Cl. C12N 5/00

(52) U.S. Cl. CPC C12N 5/0068; C12N 2533/74; C12N 2533/54

(57) ABSTRACT

The present invention relates to a method of generating a hydrogel comprising alginate dialdehyde (ADA) and collagen, which are covalently cross-linked, and optionally, further component(s), and to uses of such hydrogel. The present invention further relates to using the hydrogel for culturing cells, in particular neuronal cells, and for further uses, such as 3D bioprinting. The present invention furthermore relates to a cell culture system comprising a hydrogel of alginate dialdehyde (ADA) and collagen, which are covalently cross-linked, and, optionally, further components. Furthermore, the present invention relates to a method of generating a three-dimensional (3D) cell culture using a hydrogel according to the invention.

The present invention relates to a cell culture system comprising a hydrogel, wherein said hydrogel comprises alginate dialdehyde (ADA) and collagen, which are covalently cross-linked, and optionally, further component(s). The present invention further relates to using the cell culture system for culturing cells, in particular neuronal cells, and for further uses, such as 3D bioprinting. The present invention furthermore provides a method of generating a hydrogel of alginate dialdehyde (ADA) and collagen, which are covalently cross-linked.

### BACKGROUND OF THE INVENTION

The search for suitable three-dimensional (3D) scaffolds analogous to the natural extracellular matrices is a central task in the field of biomedical research. As two-dimensional cell culture systems are limited to imitate the structural and functional characteristics of a tissue, appropriate three-dimensional culture systems become more and more important. The adequate mechanical and chemical material properties, such as porosity and concentration of growth factors, are important key factors for natural cell-cell signaling and cell growth. Current methods for neural organoids are mostly based on Matrigel, a commercially available expensive hydrogel. Its poorly defined extracellular matrix substance, secreted by Engelbreth-Holm-Swarm mouse sarcoma cells, and it's non-adjustable, permanent stiffness are limiting its use for regenerative medicine applications.

Liu et al. (2018) review the development of collagen-based materials and describe crosslinking methods. Xu et al. (2013) describe a biological tissue fixed by alginate dialdehyde (ADA), wherein the ADA is crosslinked with decellularized porcine aorta tissue. Zhu et al. (2017) describe ADA crosslinked collagen solutions and their rheological properties; for the solution pepsin-soluble collagen from grass carp origin was used and ADA obtained by using sodium alginate from alginate (Na-ALG; viscosity: 495.0 cps at 25° C.) from Zhejiang Jingyan Biotechnology Co. LTD (China). Sarker et al., 2014 describe the fabrication of alginate-gelatin (supplier Sigma) crosslinked hydrogel microcapsules which can be used for tissue engineering.

The research on neurodegenerative diseases like Alzheimer's is an area whose progress will play a decisive role in the further development of mankind. The number of patients in our aging society is increasing and their care represents an enormous burden for the relatives and the health system. The disease mechanism of Alzheimer's disease has so far been only partially understood and translational research in this field is clearly lagging behind progress in other major problem areas such as HIV or cancer. This is partly due to the fact that examinations of the central nervous system in humans are much more difficult than, for example, research on blood or cancer tissue, which can easily be removed and examined, and where ethical problems are much less severe. Animal models, which are still the gold standard for medical, pharmacological and toxicological studies and in vitro models are therefore all the more important, but they are also associated with many difficulties. What is needed is an experimental model that can imitate the signaling cascades of cell interactions.

Thus, there is a need in the art for improved means and methods for 3D culturing of cells, in particular for neuronal cells, such as neuronal stem cells.

### SUMMARY OF THE INVENTION

According to the present invention this object is solved by a cell culture system comprising
(i) a hydrogel,
   wherein said hydrogel comprises alginate dialdehyde (ADA) and collagen, wherein the ADA and the collagen are covalently cross-linked,
   and
(ii) optionally, further component(s).

According to the present invention this object is solved by using the cell culture system of the present invention for culturing
neuronal cells, including human neuronal stem cells, hippocampus cells, dorsal root and trigeminal ganglion cells,
bone cells, including osteoblasts, osteocytes, osteoclasts,
stem cells, including pluripotent stem cells, mesenchymal stem cells, adipose derived stem cells,
immortal cell lines,
muscle cells, including myoblasts,
cartilage cells, including chondrocytes of human nasal, hyaline and fibrous cartilage,
cells forming blood vessels, fibroblasts, pericytes and endothelial cells, or
cancer tissue including epithelial cells and fibroblasts origin.

According to the present invention this object is solved by using the cell culture system of the present invention for 3D bioprinting.

According to the present invention this object is solved by using the cell culture system of the present invention as an in vitro 3D cell culture platform, preferably for drug screening and/or evaluation.

According to the present invention this object is solved by using the cell culture system of the present invention for creating tumor models.

According to the present invention this object is solved by using the cell culture system of the present invention as basis for a “lab on a chip” device.

According to the present invention this object is solved by a method of generating a hydrogel of oxidized alginate covalently crosslinked with collagen (ADA-Col), the method comprising
(1) providing alginate dialdehyde (ADA), which is obtained by controlled oxidation of sodium alginate from brown algae with sodium metaperiodate, in the absence of light, over a time period of about 2 to 10 hours, preferably about 3 to 8 hours, more preferably about 6 hours,
(2) dissolving the ADA of step (1) in a cell culture medium,
(3) adding collagen to the dissolved ADA of step (2), and furthermore adding sodium bicarbonate,
(4) obtaining the ADA-Col hydrogel.

According to the present invention the object is also solved by a method of generating a three-dimensional (3D) cell culture, said method comprising the steps:
performing the method of generating a hydrogel according to the present invention,
adding cells after step (3), and prior to or concomitantly with step (4), such that said cells become embedded in said hydrogel,
optionally further comprising, incubating said cells embedded in said hydrogel for a period in the range of from 1 h to 10 days, preferably at a temperature in the range of from 30° to 37° C.

### DESCRIPTION OF THE PREFERRED EMBODIMENTS OF THE INVENTION

Before the present invention is described in more detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art. For the purpose of the present invention, all references cited herein are incorporated by reference in their entireties.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “0.1 to 20” should be interpreted to include not only the explicitly recited values of 0.1 to 20, but also include individual values and sub-ranges within the indicated range. Thus, included in this numerical range are individual values such as 0.1, 0.2, 0.3, 0.4, 0.5, 0.6, 0.7, 0.8, 0.9, 1, 1.1 . . . 19.6, 19.7, 19.8, 19.9, 20 and sub-ranges such as from 1 to 10, 0.5 to 5, etc. This same principle applies to ranges reciting only one numerical value, such as “at least 90%”. Furthermore, such an interpretation should apply regardless of the breadth of the range or the characteristics being described.

Cell Culture System Comprising ADA-Col Hydrogel

As outlined above, the present invention provides a cell culture system comprising
(i) a hydrogel,
   and
(ii) optionally, further component(s).

(i) ADA-Col Hydrogel

The hydrogel comprised in the cell culture system comprises
(a) alginate dialdehyde (ADA) and
(b) collagen,

The ADA and the collagen are covalently cross-linked.

(a) Alginate Dialdehyde (ADA)

The ADA is obtained from sodium alginate from brown algae.

Alginate is the most abundant marine biopolymer. It exists as the most abundant polysaccharide in the brown algae comprising up to 40% of the dry matter. It is located in the intercellular matrix as a gel containing sodium, calcium, magnesium, strontium and barium ions. Alginate is widely used in industry because of its ability to retain water, and its gelling, viscosifying and stabilising properties.

Alginate is a polysaccharide derived from brown seaweed known as Phaeophyceae, considered to be a (1->4) linked polyuronic, containing three types of block structure: M block (β-D-mannuronic acid), G block (poly α-L-guluronic acid), and MG block (containing both polyuronic acids).

According to the invention, the source of the sodium alginate is important. The invention preferably uses sodium alginate (sodium alginate (E401)) from brown algae,

preferably sodium alginate from brown algae DuPont GRINDSTED® Alginate (PH 124), which is commercially available, such as from Sweet Ingredients GmbH, Germany: DuPont Pharma Alginate GRINDSTED® Alginate PH 124, viscosity (1%, 20° C., Brookfield) 250-350 mPa*s, particle sizes (95% through) max. 5%>620 μm).

In preferred embodiment, the ADA is obtained or generated by controlled oxidation of the sodium alginate with a suitable oxidizing agent, such as sodium metaperiodate (NaIO₄), potassium permanganate, or 2,2,6,6-tetramethylpiperidinyloxyl (TEMPO).

The preferred conditions for said reaction are:
absence of light,
a time period of about 2 to 10 hours, preferably about 3 to 8 hours, more preferably about 6 hours 6 hours,
in an ethanol water mixture of 50/50 (volume/volume).

The reaction is preferably supplemented with radical scavengers, such as isopropanol, during the synthesis,

The reaction is preferably quenched by the addition of ethylene glycol.

The solution is preferably dialyzed after the reaction, until periodate can no longer be determined/is absent. The ADA solution is then preferably lyophilized to obtain a white cotton-like powder product or cotton-like fleece.

The ADA can also be obtained by precipitation with isopropanol followed by centrifugation.

(b) collagen

In a preferred embodiment, the collagen is collagen type I.

(c) Obtaining the Hydrogel

For obtaining or generating the hydrogel, the ADA is dissolved in a cell culture medium before the addition of the collagen to said cell culture medium (in which the ADA is dissolved).

In the prior art, the ADA is usually dissolved in water or PBS. According to the present invention, the ADA is dissolved in a cell culture medium. The cell culture medium can be, for example,
Dulbecco's Modified Eagle Medium (DMEM) containing supplements, such as ascorbic acid (AA), insulin, transferrin, sodium selenite (ITS), serum protein, for example fetal calf serum (FCS), fetal bovine serum (FBS), horse serum (HS), dependent on the common state of the art in the respective cell culture of the target cells of interest as described herein (neuronal bone, neuronal, cartilage, etc

For example, Gibco™ Opti-MEM™ I Reduced Serum Media can be used, which is a modification of Eagle's Minimum Essential Media, buffered with HEPES and sodium bicarbonate, and supplemented with hypoxanthine, thymidine, sodium pyruvate, L-glutamine, trace elements, and growth factors. Another example is applying Opti-MEM™ Reduced Serum powder.

According to the invention, the method of obtaining or generating the ADA-Col hydrogel is important.

Typically, ADA and collagen are added to said cell culture medium, and only thereafter, a hydrogel is allowed to form.

In a preferred embodiment, the pH value of the cell culture medium is adjusted to a pH in the range from about 7.8 to 8.6, more preferably about 8.0 to 8.4, more preferably to a pH of about 8.2, before the addition of ADA and/or collagen,

and/or the temperature is in the range from 0 to 4° C., preferably about 4° C.

In one embodiment, the resultant hydrogel is a homogenous alginate dialdehyde/collagen hydrogel.

In one embodiment, in said hydrogel, said alginate dialdehyde (ADA) forms part of the bulk matrix of said hydrogel.

In one embodiment, said hydrogel is not a collagen hydrogel which has been crosslinked with ADA only after formation of a collagen hydrogel.

In one embodiment, said hydrogel is a hydrogel that has only formed after ADA and collagen have been mixed, that is the hydrogel only forms in the presence of both ADA and collagen.

The hydrogel has adjustable physico-chemical and mechanical properties, such as
hydrogel stiffness,
crosslinking density,
crosslinking degree,
diffusity,
porosity,
swelling kinetics,
degradation kinetics,
scaffold geometry,
hydrogel stress relaxation,
and/or
controllable adhesion.

In a preferred embodiment, the stiffness of the hydrogel is in the range from about 0.1 to 20 kP, preferably from about 1 to 10 kPa, preferably for culturing neuronal cells.

The hydrogel stiffness can be adjusted by final hydrogel concentrations of ADA and collagen (%) and/or the ADA synthesis conditions (such as the degree of oxidation) to meet target tissue values (bone, muscle, cartilage, . . . ) dependent on the cells that are to be cultured in the cell culture system.

(ii) Further Component(s)

The further component(s) of the cell culture system of the invention is/are preferably selected from:
growth factor(s),
antibiotic(s),
cytokine(s),
nutrient(s),
blood serum(s), such as FCS, HS
cell fragments,
saline(s) containing divalent cations, such as Ca²⁺, Mg²⁺, Ba²⁺, Sr²⁺, Cu²⁺, and/or buffer containing physiological concentrations of calcium,
glycosaminoglycan(s) supplements,
and further components of the native extracellular matrix.

In a preferred embodiment, growth factor(s) are the further component(s).

The growth factor(s) are selected dependent on the cells that are to be cultured in the cell culture system.

Preferably, the concentration of the growth factor(s) added is adjustable or adaptable to the desired application of the cell culture system.

In one embodiment, the cell culture system further comprises cells which are embedded in said hydrogel.

In one embodiment, said cells form a three-dimensional (3D) cell culture in said hydrogel.

In one embodiment, said cells are selected from
neuronal cells, including human neuronal stem cells, hippocampus cells, dorsal root and trigeminal ganglion cells,
bone cells, including osteoblasts, osteocytes, osteoclasts,
stem cells, including pluripotent stem cells, mesenchymal stem cells, adipose derived stem cells,
immortal cell lines,
muscle cells, including myoblasts,
cartilage cells, including chondrocytes of human nasal, hyaline and fibrous cartilage,
cells forming blood vessels, fibroblasts, pericytes and endothelial cells, or
cancer tissue including epithelial cells and fibroblasts origin.

Uses of the Cell Culture System

As outlined above, the present invention provides the use of the cell culture system of the present invention for culturing cells.

The cells which can be cultured in the cell culture system of the present invention are preferably selected from
neuronal cells, including human neuronal stem cells, hippocampus cells, dorsal root and trigeminal ganglion cells,
bone cells, including osteoblasts, osteocytes, osteoclasts,
stem cells, including pluripotent stem cells, mesenchymal stem cells, adipose derived stem cells,
immortal cell lines,
muscle cells, including myoblasts,
cartilage cells, including chondrocytes of human nasal, hyaline and fibrous cartilage,
cells forming blood vessels, fibroblasts, pericytes and endothelial cells, or
cancer tissue including epithelial cells and fibroblasts origin.

As outlined above, the present invention provides the use of the cell culture system of the present invention for 3D bioprinting.

As outlined above, the present invention provides the use of the cell culture system of the present invention as an in vitro 3D cell culture platform, preferably for drug screening and/or evaluation.

As outlined above, the present invention provides the use of the cell culture system of the present invention for creating tumor models.

As outlined above, the present invention provides the use of the cell culture system of the present invention as basis for a “lab on a chip” device.

The cell culture system can be used as the basis or fundament or substrate for a “lab on a chip” device, which is a miniaturized device that integrates onto a single chip one or several analyses, which are usually done in a laboratory; analyses such as DNA sequencing or biochemical detection. Research on lab-on-a-chip usually focuses on diagnostics and analysis.

Method of Generating ADA-Col Hydrogels

As outlined above, the present invention provides a method of generating a hydrogel of oxidized alginate covalently crosslinked with collagen (ADA-Col).

The method comprises
(1) providing alginate dialdehyde (ADA),
(2) dissolving the ADA of step (1) in a cell culture medium,
(3) adding collagen to the dissolved ADA of step (2), and furthermore adding sodium bicarbonate to said cell culture medium,
(4) obtaining the ADA-Col hydrogel.

In step (1), an alginate dialdehyde (ADA) is provided.

The ADA is obtained by controlled oxidation of sodium alginate from brown algae, as it is described above, with a suitable oxidizing agent, such as sodium metaperiodate (NaIO₄), potassium permanganate, or 2,2,6,6-tetramethylpiperidinyloxyl (TEMPO).

The preferred conditions for said reaction are:
absence of light,
a time period of about 2 to 10 hours, preferably about 3 to 8 hours, more preferably about 6 hours 6 hours,
in a mixture of ethanol and water of 50/50 (volume/volume).

The reaction is preferably supplemented with radical scavengers, such as isopropanol, during the synthesis,

The reaction is preferably quenched by the addition of ethylene glycol.

The solution is preferably dialyzed after the reaction, until periodate can no longer be determined/is absent. The ADA solution is then preferably lyophilized to obtain a white cotton-like powder product or cotton-like fleece.

The ADA can also be obtained by precipitation with isopropanol followed by centrifugation.

In step (2), the pH value of the cell culture medium is preferably adjusted to a pH from about 7.8 to 8.6, more preferably 8.0 to 8.4, more preferably to a pH of about 8.2, before the addition of ADA and/or collagen.

In step (3), the collagen added is preferably collagen type I.

In step (3), the temperature is preferably in the range from 0 to 4° C., preferably about 4° C. The present invention also relates to a method of generating a three-dimensional (3D) cell culture, said method comprising the steps:
performing the method of generating a hydrogel according to the present invention,
adding cells after step (3), and prior to or concomitantly with step (4), such that said cells become embedded in said hydrogel,
optionally further comprising, incubating said cells embedded in said hydrogel for a period in the range of from 1 h to 10 days, preferably at a temperature in the range of from 30° C. to 37° C.

In one embodiment said cells are selected from:
neuronal cells, including human neuronal stem cells, hippocampus cells, dorsal root and trigeminal ganglion cells,
bone cells, including osteoblasts, osteocytes, osteoclasts,
stem cells, including pluripotent stem cells, mesenchymal stem cells, adipose derived stem cells,
immortal cell lines,
muscle cells, including myoblasts,
cartilage cells, including chondrocytes of human nasal, hyaline and fibrous cartilage,
cells forming blood vessels, fibroblasts, pericytes and endothelial cells, or
cancer tissue including epithelial cells and fibroblasts origin.

### PREFERRED EMBODIMENTS

Research with human neuronal stem cells in a soft 3D hydrogel system is of great importance. Hydrogels are hydrophilic polymers of natural or synthetic origin. The appropriate hydrogels for this application should exhibit controllable swelling and degradation kinetics, as well as adjustable mechanical properties, tailored chemical and physical structure, crosslinking density, diffusivity and porosity. Especially, the supply of oxygen and nutrients throughout the hydrogel depends on the porosity, pore diameter and pore interconnectivity, which are decisive parameters affecting also cell growth and proliferation in the 3D matrix.

Various hydrogels have recently been used to mimic the extracellular matrix of several tissues; however, the adaptation of material properties and scaffold geometry for tissue engineering remains a challenge. Matrigel is an established hydrogel for three-dimensional cell culture. It consists of a protein mixture extracted from a soft tissue tumor of the mouse. As a result, the contained protein concentrations and the stiffness vary immensely from batch to batch. However, cell behavior in culture is strongly dependent on these factors.

In contrast, in the here described newly established hydrogel, stiffness and concentrations of growth factors can be reproducibly adapted, such as to neuronal growth. This novel hydrogel system based on oxidized alginate covalently crosslinked with collagen (ADA-Col) can be utilized to design neuronal network constructs, in which cell growth, proliferation and migration can be observed in detail for an extended period. As a result the neuronal network growing in our new tissue culture system is clearly more similar to the natural neural tissue as any other culture yet published. Besides, with this new in vitro system we contribute to the ethic requirements in animal research by replacement, refinement and reduction (“three Rs”).

In conclusion, we have invented a system for three-dimensional cell culture with controlled swelling and degradation kinetics, as well as adjustable mechanical properties, tailored chemical and physical structure, crosslinking density, diffusivity and porosity, adaptable material properties such as hydrogel stiffness and adaptable scaffold geometry, variably adaptable growth factors. This avoids batch to batch variations and allows high reproducibility.

In addition, the hydrogel of the invention is significantly cheaper than the commercially available Matrigel hydrogel. Another major advantage is the three-dimensional self-organization of the cells within the hydrogel. This behavior could be reproducibly proven in the cultivation of dorsal root ganglion cells. The self-organization to ball-like structures is very similar to the real structure in animals. This self-organization is a clear sign that the hydrogel provides a three-dimensional matrix for the cells, which does not significantly change grows and cell physiology.

The following examples and drawings illustrate the present invention without, however, limiting the same thereto.

### BRIEF DESCRIPTION OF THE DRAWINGS

FIG. 1. Culturing neuronal cells in 2D versus 3D culture.

A) 2D cell culture of neuronal cells results in forced basal-apical cell polarity, wrong stiffness and porosity.

B) 3D cell culture system allows free cell polarity.

FIG. 2. Preparation of dorsal root ganglion (DRG) cells.

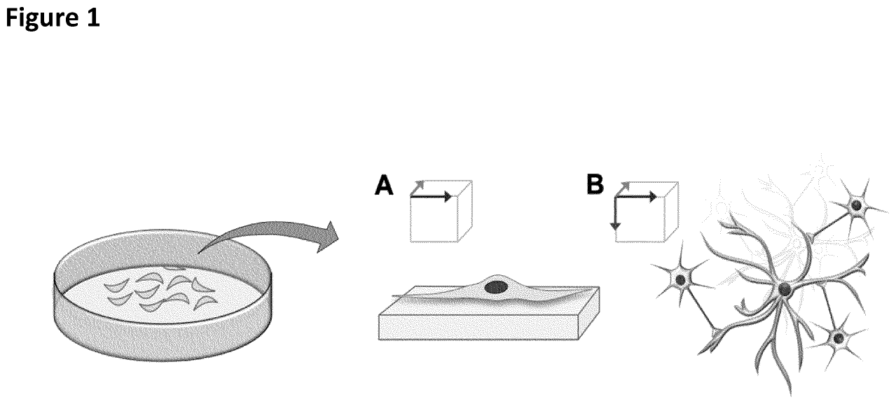

a) from sacrificed mice. b) Dousing fur with 70% ethanol. c) Foreceps were used for removing skin by using scissors with one big cut. d,e) Removing spinal cord with two long incisions along the spinal cord, then cutting hips and neck. f) Spinal cord washed in PBS then muscle, fat and other soft tissues were cut from the spinal cord. g,h) Thick forceps were used to secure the spinal column dorsal side up, before cutting it into two equal halves along the midline. i) The spinal cord was peeled from the pinned column in a rostral to caudal direction. j) Individual ganglia were extricated by clasping and lifting with forceps the distally projecting axon bundles found on the lateral side of the DRG. k,l) Care must be taken not to damage the DRG with the forceps DRG were then pinned out via their axons, and any residual meninges removed before cutting the axons close to the DRG.

FIG. 3. DRG cells grow in the ADA-Col hydrogel of the invention and show 3D self-organization within the hydrogel.

A) Live-dead staining of DRG from baby C57/b6 mice in ADA collagen hydrogel after three days of incubation at 37° C. and 5% CO2 in the incubator (top). Live-dead staining of DRG cells from baby C57BL/6 mice in ADA collagen hydrogel after seven days of incubation (bottom). Living cells stained in green with Calcein and dead cells stained in red with Propidium iodide. The scale bar is 50

B) Live-dead staining of DRG cells from baby C57/b6 mice in matrix hydrogel C57/b6 after three days incubation at 37° Cs and 5% CO2 in incubator (top). Live dead staining of DRG cells from baby C57BL/6 mice in matrix hydrogel after seven days of incubation (bottom). Living cells stained in green with Calcein and dead cells stained in red with Propidium iodide. The scale bar is 50

FIG. 4. Neuronal cells grow in the ADA-Col hydrogel of the invention.

Immunhistochemistry staining of DRG cells from baby C57/b6 mice in matrix hydrogel C57/b6 after three days incubation at 37° Cs and 5% CO2 in incubator (top) and after seven days of incubation (bottom). The scale bar is 50

### EXAMPLES

Example 1 Alginate Dialdehyde (ADA) Synthesis Sodium alginate (sodium alginate (E401) from brown algae, DuPont GRINDSTED Alginate (PH 124) was obtained from Sweet Ingredients GmbH, Germany (material number: 60516). Sodium metaperiodate and calcium chloride di-hydrate (CaCl₂×2H₂O) were purchased from Sigma Aldrich, Germany.

Alginate di-aldehyde (ADA) was synthesized by controlled oxidation of sodium alginate in a mixture of equal volumes of ethanol and water. Briefly, 10 g of sodium alginate PH 124 were dispersed in 50 ml of ethanol (Sigma Aldrich, Germany) and 2.674 g of sodium metaperiodate were dissolved in 50 ml of ultrapure water (Direct-Q, Merck Millipore, Germany) in the absence of light to get a 12.5 mmol sodium metaperiodate solution. The periodate solution was slowly added to the sodium alginate dispersion, which was continuously stirred at 250 300 rpm in the dark at 22° C. (room temperature) for 6 hours. The reaction was quenched after 6 hours by adding 10 ml of ethylene glycol (density 1.113 g·ml⁻¹ at 25° C.) (Sigma Aldrich, Germany) under continuous stirring for 30 minutes. The resultant suspension was dialyzed against ultrapure water (Direct-Q®, Merck Millipore, Germany) using a dialysis membrane with a molecular weight cut off (MWCO) of 6000-8000 Da (Repligen Biotech, Spectrumlabs, USA) for 5 days with water changes twice a day. The absence of periodate was confirmed by adding 0.5 ml of 1% (w/v) silver nitrate (Sigma Aldrich, Germany) solution to 0.5 ml of ADA ensuring the absence of any precipitate. The final ADA solution was frozen at −21° C. for a minimum of 24 hours and lyophilized using a freeze dryer (Alpha 1-2 LD plus, Martin Christ, Germany) for one week.

### Example 2 Cell Preparation

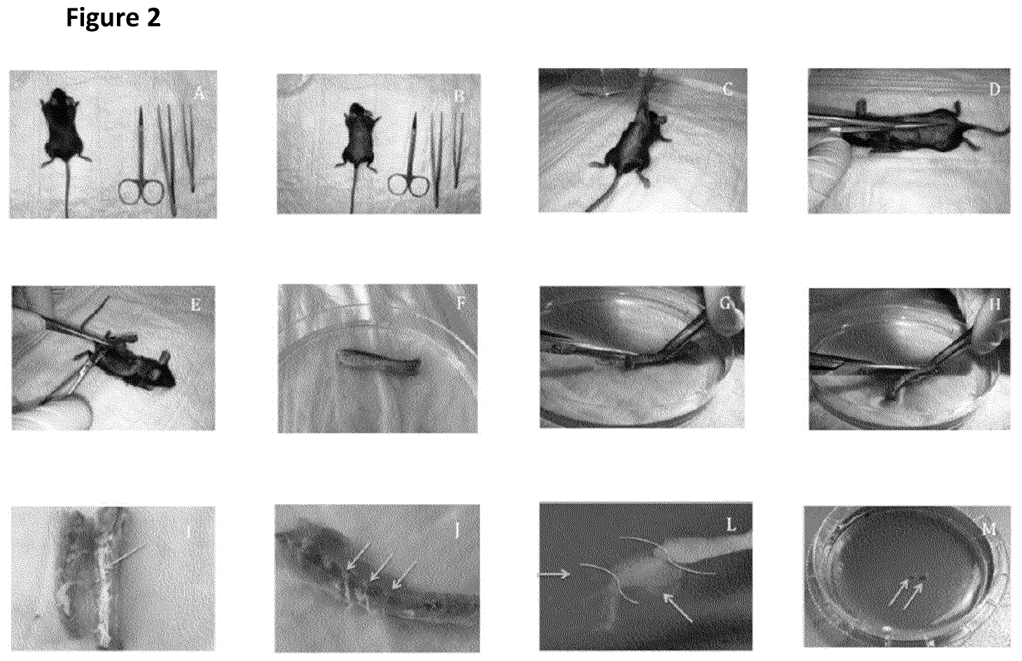

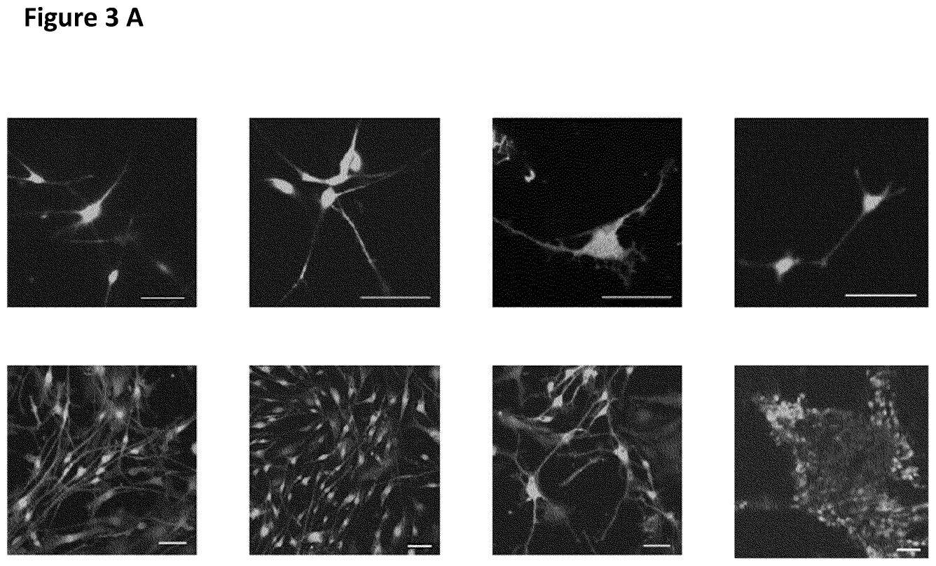

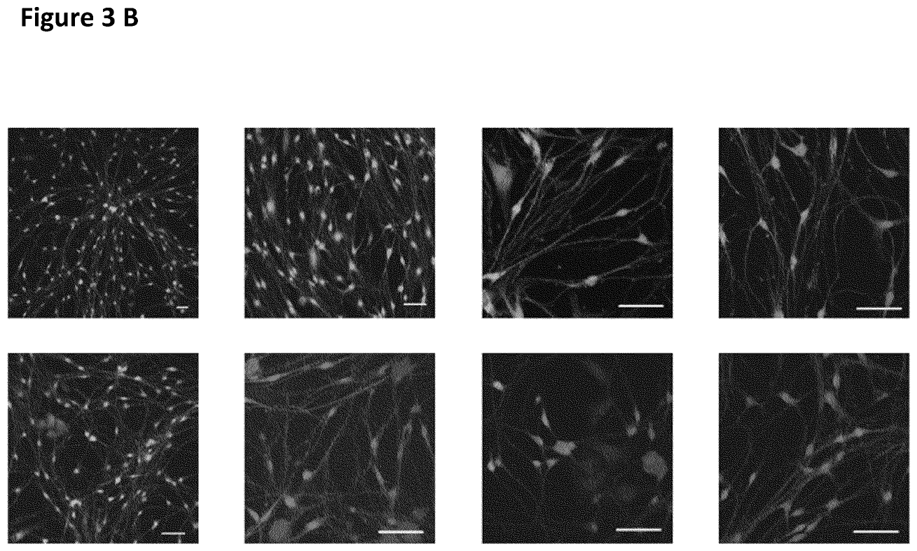

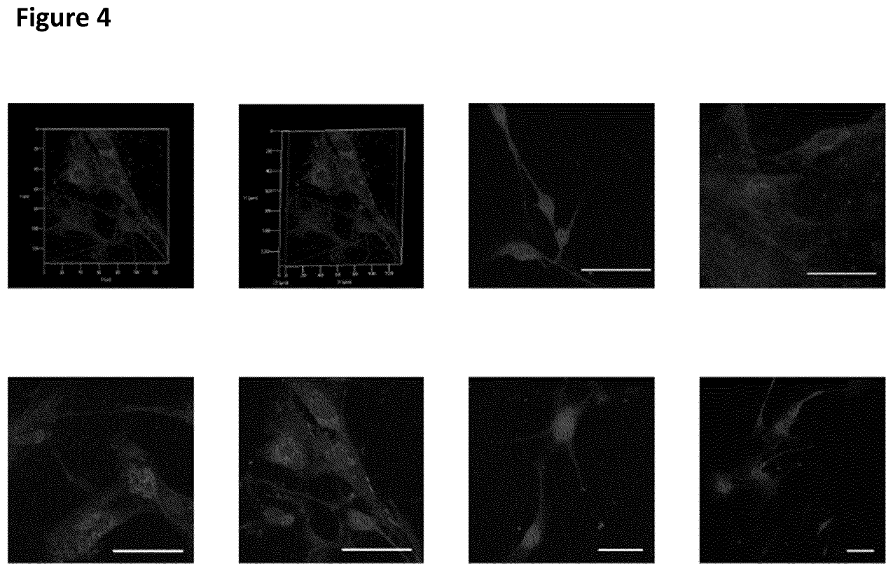

Dorsal root ganglion (DRG) cells were obtained from three to seven days old wildtype C57BL/6 mice sacrificed in carbon dioxide atmosphere to prevent damage of cervical DRGs (Sleigh, Weir, & Schiavo, 2016). The spinal cord was dissected and DRGs (20-35 of each animal) were collected in phosphate buffered saline (PBS). The cell preparation is shown in FIG. 2. Briefly, DRGs were placed into Dulbecco's Modified Eagle Medium 4.5 g/L (DMEM, Gibco, Germany), where nerve trunks and connective tissue were dissected. DMEM was removed and Enzyme mix (see Table 1) was added. Following a 30 min incubation in a humidified incubator (37° C., 5% CO2), DRG were washed with DMEM twice and once with TNB100 basal medium (TNB, Biochrom, Germany). The cell suspension was spun for 3 minutes at 1000 rpm. By triturating DRG through a glass pipette the ganglion cells were dissociated and the cell pellet was resuspended.

TABLE 1
List of chemicals and medium

| | |
|---|---|
| DMEM | 500 ml DMEM (Gibco, Germany) + |
| | 2.5 ml Gentamycin (Sigma, Germany) |
| Enzyme mix | 50 ml DMEM + |
| | 50 mg Collagenase (Sigma, Germany) + |
| | 25 mg Protease (Sigma, Germany) |
| TNB (Biochrom, | +2 ml Proteincomplex (Biochom, Germany) + |
| Germany) | 1 ml Penicillin Steptomycin (PenStrep, Sigmal, |
| | Germany) + 500 μlnerve growth factor (NGF, |
| | Alomone Labs Nr. 130, Germany) |

### Example 3 Hydrogel Preparation

For the 4× Opti-MEM medium, 13.6 g Opti-MEM reduced serum medium powder (ThermoFisher, Germany) were dissolved in 200 ml aqua dest, stirring for 20 minutes. Subsequently 2.4 g sodium hydrogen carbonate (Roth, Germany) was added. A pH value of 8.2 was adjusted finally in 250 ml aqua dest. Finally, the 4× Opti-MEM was sterilely filtered through a 0.22 μm filter (Roth, Germany).

0.1 g ADA (PH 124, Sweet Ingredients GmbH, Germany) were dissolved in 2500 μl 4× Opti-MEM under continuous stirring for 1 hour. The ADA dissolved in Optimem was filtered sterile by a 0.22 μm filter (Roth, Germany). In a 15 ml falkon (VWR, Germany) 75 μl ADA dissolved in 4× Opti-MEM, 164.4 μl Collagen type I (Corning, Germany), 4 μl sodium bicarbonate (Roth, Germany) 3 μl penicillin/streptomycin (Sigma, Germany), 53.5 μl aqua dest. and 3 μl NGF (Alomone Labs Nr. 130, Germany) were mixed on ice to a total stock solution of 300 μl in a 15 ml falkon.

The prepared DRG cells (see Example 2) were taken up in 150 μl TNB medium and vortexted with 300 μl total stock solution. 225 μl each were seeded in one Ibidi vessel (Ibidi, Germany). The hydrogel was then incubated with the cells for one hour at 37 degrees Celsius and 5% CO2 in the incubator. On each well 150 μl FCS (Gibco, Germany) with 30 μl NGF were added and then incubated at 37 degrees Celsius, 5% CO2 for 3 and 7 days.

### Example 4 Live-Dead Staining

Calcein/propidium iodide (PI, Thermofisher, Germany) iodide assay was used to estimate the ratio of live/dead cells. Using the following protocol, living cells were stained with green fluorescent marker calcein and dead cells with red propium iodide (PI). (Non-fluorescent calcein is taken up by living cells and cut intracellularly by an esterase. Afterwards, calcein is green fluorescent and impermeable for cell membrane. PI is a red fluorescent dye for nuclei, which is impermeable for cell membrane of living cells but binds diploid DNA).

Hydrogel was washed with Hank's balanced salt solution (HBSS, Sigma, Germany), followed by adding staining solution to the sample at a final concentration of 4μl/ml calcein/HBSS and 1μl/ml PI/HBSS. After 45 minutes of incubation of the sample in the dark. Before imaging the hydrogel was washed with HBSS. For imaging, live and dead cell fluorescence microscopy (Axio, Zeiss, Germany) was used.

Live-dead staining using calcein and propidium iodide showed that >99% of neurons were living. Results are shown in FIGS. 3A and B.

### Example 5 Immunohistochemistry

Immunohistochemistry was made after checking the dendrite growth with light microscopy after three and seven days of incubation. Manufacturer, details and dilutions of primary and secondary antibody are shown in Tables 2 to 4.

TABLE 2
List of chemicals

| | |
|---|---|
| 4% Paraformaldehyde (PFA) | 40 g Paraformaldehyde + |
| | 500 ml Aqua dest + |
| | 14.42 g NA₂HPO₄x2H₂O |
| PBS-Bovine serum albumin (PBS-BSA) | 50 ml PBS + |
| | 0.5 g BSA (Sigma, Germany) |
| 0.5% TritonX-100 (PBS-BSA-TX) | 100 ml PBS-BSA + |
| | 0.5 g TritonX (Sigma, Germany) |
| 4′,6′-Diamidino-2-phenylindole | |
| hydrochloride (DAPI) | |

TABLE 3
List of primary antibodies (dissolved in PBS/BSA/TX)

| | | Antigen | | |
|---|---|---|---|---|
| Name | Host | Characteristics | Source | Dilution |
| Guinea Pig | Guinea pig | Soluble | Chemicon | 1:50 |
| Anti-Protein | | cytoplasmic | international, | |
| gene product | | human PGP 9.5 | USA | |
| 9.5 (GP PGP | | | | |
| 9.5) | | | | |
| Anti- | Rabbit | Phosporylated | Sigma, | 1:200 |
| Neurofilament | | H tail of | Germany | |
| 200 | | Neurofilament | | |

TABLE 4
List of secondary antibodies (dissolved in PBS/BSA/TX)

| | | | | |
|---|---|---|---|---|
| | | Antigen | | |
| Name | Host | Characteristics | Source | Dilution |

| | | | |
|---|---|---|---|
| Cy3-AffiniPure | Donkey Anti- | GP PGP 9.5 | Jackson |
| | guinea pig IgG | | Immuno |
| | (H + L) | | Research, |
| | | | USA |
| Donkey anti- | Donkey anti | Alexa 488 | Thermo |
| goat IgG | goat IgG | | Fisher, |
| | | | USA |

Hydrogel was fixed with 4% (w/v) paraformaldehyde (PFA, pH 7.4, Sigma, Germany) for 10 minutes, followed by washing two times for 10 minutes in PBS and incubated for “blocking” with 5% donkey normal serum in PBS-BSA-TX overnight. (Triton X100 is increasing the antibody permeability and blocking serum is used to minimize nonspecific bindings to the surfaces).

After blocking, hydrogel was washed for 10 minutes in PBS followed by incubation with guinea pig anti-protein gene product 9.5 (GP PGP 9.5, Chemicon International, USA) antibody or Anti-Neurofilament200 (Sigma, Germany) in PBS-BSA-TX. After overnight incubation of the primary antibody at room temperature, 3 washes with PBS (15 minutes each) were performed, followed by addition of the secondary antibody Cy3-AffinPure donkey anti-guinea pig (Chemicon international, USA) and 4′,6′-diamidino-2-phenylindole hydrochloride (DAPI, Sigma-Aldrich, USA). After 4 h of incubation with the secondary antibodies, hydrogel was finally washed three times in PBS.

Confocal microscopy was used for imaging of both live and fixed samples. The immunostained samples were analysed using a LSM 780 light and confocal microscope (Carl Zeiss MicroImaging GmbH, Jena, Germany) mounted on an inverted Axio Observer Z1. Three dry objective lenses (10×, 20× and 40×) were used. Fluorescent structures were observed in the light path mode using red and green filters. Confocal images were taken using filter settings for Alexa 488 and 555 with a resolution of 1024×1024 or 512×512 pixels. Z-stacks of images were taken to approve the 3D-growth of ganglion cells. Pictures were converted to a 12-bit RGB tiff-file using confocal assistant software ZEN 2010. After 72 and 168 hours (three to seven days) the cultured ganglion cells showed 2-5 extensions that formed a dense three-dimensional network after three days (FIG. 4, top) and seven days (FIG. 4, bottom). The cells consisted mainly of neurons, glial cells could not clearly be identified. Live-dead staining using calcein and propidium iodide showed that >99% of neurons were living (Example 4).

### REFERENCES

Liu X, Zheng C, Luo X, Wang X, Jiang H. Materials Science & Engineering C Recent advances of collagen-based biomaterials: Multi-hierarchical structure, modification and biomedical applications. Mater Sci Eng C. 2019; 99 (June 2018):1509-1522. doi:10.1016/j.msec.2019.02.070
Sarker B, Papageorgiou D G, Silva R, et al. Fabrication of alginate-gelatin crosslinked hydrogel microcapsules and evaluation of the microstructure and physico-chemical properties. J Mater Chem B. 2014; 2(11):1470. doi:10.1039/c3tb21509a
Xu Y, Huang C, Li L, et al. In vitro enzymatic degradation of a biological tissue fixed by alginate dialdehyde. Carbohydr Polym. 2013; 95(1):148-154. doi:10.1016/j.carbpol.2013.03.021
Zhu S D, Yu X, Xiong S, et al. Insights into the rheological behaviors evolution of alginate dialdehyde crosslinked collagen solutions evaluated by numerical models. Mater Sci Eng C. 2017; 78: 727-737. doi:10.1016/j.msec.2017.04.125

1. A cell culture system comprising:
(i) a hydrogel,
wherein said hydrogel comprises alginate dialdehyde (ADA) and collagen, wherein the ADA and the collagen are covalently cross-linked,
and
(ii) optionally, further component(s).

2. (canceled)

3. The cell culture system of claim 1, wherein the hydrogel is obtained by dissolving ADA in a cell culture medium before adding the collagen to said cell culture medium,
wherein the pH of the cell culture medium is from 8.0 to 8.4, before the addition of ADA and/or collagen,
and/or wherein the temperature is from 0 to 4° C.

4. The cell culture system according to claim 1, wherein the collagen is collagen type I.

5. The cell culture system according to claim 1, wherein the hydrogel has adjustable:
hydrogel stiffness,
crosslinking density,
crosslinking degree,
diffusity,
porosity,
swelling kinetics,
degradation kinetics,
scaffold geometry,
hydrogel stress relaxation,
and/or
controllable adhesion.

6. The cell culture system according to claim 1, comprising one or more further components selected from:
growth factor(s),
antibiotic(s),
cytokine(s),
nutrient(s),
blood serum(s),
cell fragments,
saline containing divalent cations, and/or buffer containing physiological concentrations of calcium,
glycosaminoglycan(s) supplements, and/or
further components of native extracellular matrix.

7. The cell culture system according to claim 1, further comprising cells that are embedded in said hydrogel.

8. The cell culture system of claim 7, wherein said cells form a three-dimensional (3D) cell culture in said hydrogel.

9. The cell culture system according to claim 7, wherein said cells are selected from
neuronal cells,
bone cells,
stem cells,
immortal cell lines,
muscle cells,
cartilage cells,
cells forming blood vessels, and
cancer cells.

10. A method for culturing cells, wherein said method comprises the use of the cell culture system of claim 1 and wherein the cells are selected from
neuronal cells,
bone cells,
stem cells,
immortal cell lines,
muscle cells,
cartilage cells,
cells forming blood vessels, and
cancer cells.

11. A method for 3D bioprinting, wherein said method comprises the use of a cell culture system of claim 1.

12. Use of the cell culture system of claim 1 as an in vitro 3D cell culture platform.

13. A method for creating a tumor, wherein said method comprises use of the cell culture system of claim 1.

14. The cell culture system of claim 1 used to create a “lab on a chip” device.

15. A method of generating a hydrogel of oxidized alginate covalently crosslinked with collagen (ADA-Col), the method comprising:
(1) providing alginate dialdehyde (ADA), which is obtained by controlled oxidation of sodium alginate from brown algae with an oxidizing agent, in the absence of light, over a time period of about 2 to 10 hours,
(2) dissolving the ADA of step (1) in a cell culture medium,
(3) adding collagen to the dissolved ADA of step (2), and furthermore adding sodium bicarbonate to said cell culture medium,
(4) obtaining the ADA-Col hydrogel.

16. The method of claim 15, wherein during obtaining the ADA provided in step (1),
the reaction is in a mixture of ethanol and water (50/50 volume/volume),
and/or supplemented with radical scavengers during the synthesis,
and/or wherein the reaction is quenched by the addition of ethylene glycol.

17. The method of claim 15, wherein the pH of the cell culture medium is about 7.8 to 8.6, before the addition of ADA and/or collagen.

18. The method of claim 15, wherein the temperature of step (3) is from 0 to about 4° C.

19. A method of generating a three-dimensional (3D) cell culture, said method comprising the steps:
performing the method of generating a hydrogel according to claim 15,
adding cells after step (3), and prior to, or concomitantly with, step (4), such that said cells become embedded in said hydrogel,
optionally further comprising, incubating said cells embedded in said hydrogel for a period of from 1 h to 10 days.

20. The method according to claim 19, wherein said cells are selected from:
neuronal cells,
bone cells,
stem cells,
immortal cell lines,
muscle cells,
cartilage cells,
cells forming blood vessels, and
cancer cells.

21. The method according to claim 15, wherein the oxidizing agent is selected from sodium metaperiodate, potassium permanganate, and 2,2,6,6-tetramethylpiperidinyloxyl (TEMPO).
